(19) United States
(12) Patent Application Publication
(10) Pub. No.: US 2024/0065565 A1
(43) Pub. Date: Feb. 29, 2024

(54) METHODS, SYSTEMS, AND STORAGE MEDIUMS FOR MONITORING HEART RATE

(71) Applicant: SHENZHEN SHOKZ CO., LTD., Shenzhen (CN)

(72) Inventors: Xin ZHOU, Shenzhen (CN); Fengyun LIAO, Shenzhen (CN); Xin QI, Shenzhen (CN)

(73) Assignee: SHENZHEN SHOKZ CO., LTD., Shenzhen (CN)

(21) Appl. No.: 18/505,071

(22) Filed: Nov. 8, 2023

Related U.S. Application Data
(63) Continuation of application No. PCT/CN2021/117467, filed on Sep. 9, 2021.

(51) Int. Cl. A61B 5/024; A61B 5/00; A61B 5/11

(52) U.S. Cl. CPC A61B 5/02416; A61B 5/11; A61B 5/721; A61B 5/7235; A61B 2562/0219; A61B 2562/0233

(57) ABSTRACT

The present disclosure provides a method, system, and readable medium for monitoring a heart rate. The method may include: obtaining a first signal, the first signal including a target heart rate signal in a motion state; obtaining a motion signal corresponding to the motion state; identifying a second signal with a target frequency from the first signal based on a motion frequency corresponding to the motion signal, the target frequency originating from a linear superposition of the motion frequency and a heart rate frequency corresponding to the target heart rate signal; and determining the target heart rate signal by processing, based on the motion signal and the second signal, the first signal.

### CROSS-REFERENCE TO RELATED APPLICATIONS

This application is a continuation of International Application No. PCT/CN2021/117467, filed on Sep. 9, 2021, and the entire contents of which are hereby incorporated by reference.

### TECHNICAL FIELD

The present disclosure relates to data monitoring field, and in particular, to methods, systems, and storage mediums for monitoring a heart rate.

### BACKGROUND

With a popularization of smart wearable devices, electronic devices with heart rate monitoring functions such as watches, bracelets, etc. become increasingly popular among users. During a heart rate monitoring process, a collected heart rate signal may include a motion artifact (MA) signal due to interferences from the motion and other factors. A “clean” (i.e., with a relatively high signal-to-noise ratio) heart rate signal may be obtained by removing, from the collected heart rate signal, the MA signal through signal processing processes. However, there may be a coupling relationship, such as a superimposed relationship, between the MA signal and the heart rate signal, which may affect the accuracy of the heart rate monitoring process.

Therefore, it is desirable to provide methods for monitoring a heart rate to process the collected heart rate signal based on superimposed characteristics between the MA signal and the heart rate signal, thereby improving the accuracy of a heart rate monitoring result.

### SUMMARY

One aspect of the present disclosure provides a method for monitoring a heart rate. The method may include: obtaining a first signal, the first signal including a target heart rate signal in a motion state; obtaining a motion signal corresponding to the motion state; identifying a second signal with a target frequency from the first signal based on a motion frequency corresponding to the motion signal, the target frequency originating from a linear superposition of the motion frequency and a heart rate frequency corresponding to the target heart rate signal; and determining the target heart rate signal by processing, based on the motion signal and the second signal, the first signal.

In some embodiments, the obtaining a motion signal corresponding to the motion state includes: obtaining a filtered signal by performing a filtering operation on the first signal; and determining the motion signal based on the filtered signal.

In some embodiments, the obtaining a motion signal corresponding to the motion state includes obtaining the motion signal through an acceleration sensor.

In some embodiments, the obtaining a motion signal corresponding to the motion state includes: obtaining two or more first signals using two or more optical paths; and determining the motion signal based on the two or more first signals.

In some embodiments, the second signal includes a superimposed signal between the motion signal and the target heart rate signal.

In some embodiments, the superimposed signal includes a non-linear superimposed signal.

In some embodiments, the target frequency is equal to a sum of the motion frequency and the heart rate frequency.

In some embodiments, the target frequency is equal to a difference between the motion frequency and the heart rate frequency.

In some embodiments, the determining the target heart rate signal by processing, based on the motion signal and the second signal, the first signal includes: determining the target heart rate signal by removing the motion signal and the second signal from the first signal.

In some embodiments, the determining the target heart rate signal by processing, based on the motion signal and the second signal, the first signal further includes: determining a signal amplitude of the motion signal; determining whether the signal amplitude is greater than an amplitude threshold; and in response to determining that the signal amplitude is greater than the amplitude threshold, determining the target heart rate signal by processing, based on the motion signal and the second signal, the first signal.

In some embodiments, the determining the target heart rate signal by processing, based on the motion signal and the second signal, the first signal further includes: determining a signal frequency of the motion signal; determining whether the signal frequency is greater than a frequency threshold; and in response to determining that the signal frequency is greater than the frequency threshold, determining the target heart rate signal by processing, based on the motion signal and the second signal, the first signal.

In some embodiments, the target heart rate signal in the motion state included in the first signal is obtained using a photocapacitive pulse wave sensor.

One aspect of the present disclosure provides a system for monitoring a heart rate. The system includes at least one storage device storing a set of instructions; and at least one processor in communication with the storage device, wherein when executing the set of instructions, the at least one processor is directed to cause the system to: obtain a first signal, the first signal including a target heart rate signal in a motion state; obtain a motion signal corresponding to the motion state; identify a second signal with a target frequency from the first signal based on a motion frequency corresponding to the motion signal, the target frequency originating from a linear superposition of the motion frequency and a heart rate frequency corresponding to the target heart rate signal; and determine the target heart rate signal by processing, based on the motion signal and the second signal, the first signal.

One aspect of the present disclosure provides a system for monitoring a heart rate. The system includes an obtaining module, a processing module, and a generation module. The obtaining module may be configured to obtain a first signal, the first signal including a target heart rate signal in a motion state. The processing module may be configured to: obtain a motion signal corresponding to the motion state; and identify a second signal with a target frequency from the first signal based on a motion frequency corresponding to the motion signal, the target frequency originating from a linear superposition of the motion frequency and a heart rate frequency corresponding to the target heart rate signal. And the generation module may be configured to determine the target heart rate signal by processing, based on the motion signal and the second signal, the first signal.

One aspect of the present disclosure provides a non-transitory computer readable medium, including executable instructions that, when executed by at least one processor, cause the at least one processor to perform a method of the present disclosure.

Additional features of the present disclosure may be described in the following description. Through the study of the following description and corresponding drawings or the understanding of the production or operation of the embodiment, some additional features of the present disclosure are obvious to those skilled in the art. The features of the present disclosure can be realized and obtained by practice or using various aspects of the methods, tools and combinations described in the following embodiments.

### BRIEF DESCRIPTION OF THE DRAWINGS

The present disclosure is further illustrated in terms of exemplary embodiments, and these exemplary embodiments are described in detail with reference to the drawings. These embodiments are not restrictive. In these embodiments, the same number indicates the same structure, wherein:

FIG. 1 is a schematic diagram illustrating an application scenario of a system for monitoring a heart rate according to some embodiments of the present disclosure;

FIG. 2 is a schematic diagram illustrating an exemplary computing device according to some embodiments of the present disclosure;

FIG. 3 is a schematic diagram illustrating exemplary hardware and/or software components of an exemplary mobile device according to some embodiments of the present disclosure;

FIG. 4 is a block diagram illustrating an exemplary system for monitoring a heart rate according to some embodiments of the present disclosure;

FIG. 5 is a flowchart illustrating an exemplary system for monitoring a heart rate according to some embodiments of the present disclosure;

FIG. 6 is a schematic diagram illustrating an exemplary spectrum of a first signal according to some embodiments of the present disclosure;

FIG. 7 is a flowchart illustrating an exemplary process for monitoring a heart rate according to some embodiments of the present disclosure; and

FIG. 8 is a flowchart illustrating an exemplary process for monitoring a heart rate according to other embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the accompanying drawing in the following description is merely some examples or embodiments of the present disclosure, for those skilled in the art, the present disclosure may further be applied in other similar situations according to the drawings without any creative effort. Unless obviously obtained from the context or the context illustrates otherwise, the same numeral in the drawings refers to the same structure or operation.

As used in the disclosure and the appended claims, the singular forms “a,” “an,” and “the” include plural referents unless the content clearly dictates otherwise. Generally speaking, the terms “comprise” and “include” only imply that the clearly identified steps and elements are included, and these steps and elements may not constitute an exclusive list, and the method or device may further include other steps or elements.

The flowcharts used in the present disclosure illustrate operations that the system implements according to the embodiment of the present disclosure. It should be understood that a previous operation or a subsequent operation of the flowcharts may not be accurately implemented in order. Instead, a plurality of steps may be processed in reverse or simultaneously. Moreover, other operations may further be added to these procedures, or one or more steps may be removed from these procedures.

The system and method for monitoring a heart rate provided in the embodiments of the present disclosure may be described in detail below, combined with accompanying drawings.

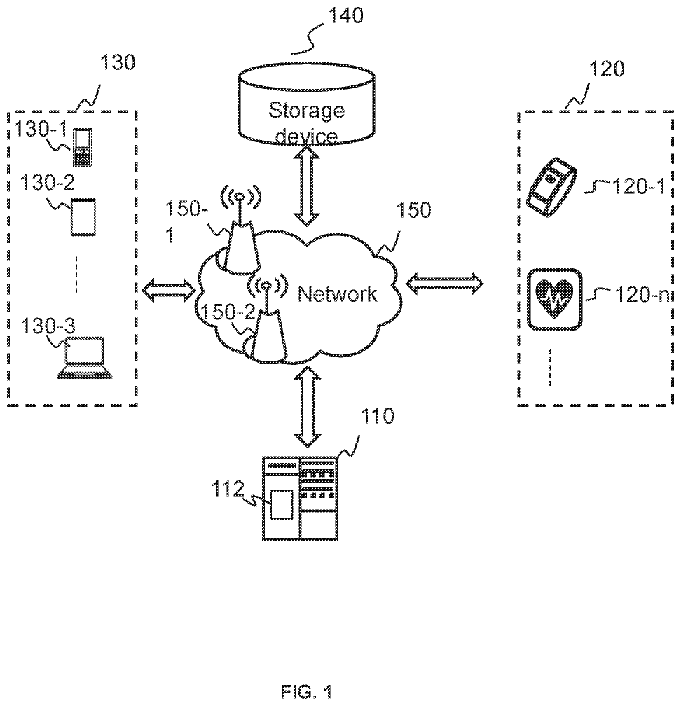

FIG. 1 is a schematic diagram illustrating an application scenario of a system for monitoring a heart rate according to some embodiments of the present disclosure. A heart rate monitoring system 100 may be applied in various software, systems, platforms, and devices to achieve heart rate signal monitoring and processing. For example, the heart rate monitoring system 100 may be applied in various software, systems, platforms, and devices to perform a noise reduction operation on a heart rate signal to remove a motion signal in the heart rate signal, thereby improving the accuracy of the heart rate signal monitored by a user in a motion state.

When the user is in the motion state, heart rate data collected by a heart rate monitoring device (e.g., a collection device 120 shown in FIG. 1) is not a clean heart rate signal, which may also include a motion signal caused by the user's motion or include a superimposed signal between the heart rate signal and the motion signal (also referred to as a second signal). In such cases, a clean heart rate signal (also referred to as a target heart rate signal) may be obtained by removing the motion signal and the second signal, thereby improving the accuracy of a result of a heart rate monitoring. A system and method for monitoring the heart rate may be provided in the embodiments of the present disclosure, which may perform the noise reduction operation on the heart rate signal in motion scenes.

As shown in FIG. 1, the heart rate monitoring system 100 may include a processing device 120, a terminal 130, a storage device 140, and a network 150.

In some embodiments, the processing device 110 may process data and/or information obtained from other devices or system components. The processing device 110 may execute program instructions based on the data, information, and/or processing results to perform one or more functions described in the present disclosure. For example, the processing device 110 may obtain a first signal of the user in a motion state and a motion signal corresponding to the motion state. As another example, the processing device 110 may identify a second signal with a target frequency from the first signal based on a motion frequency corresponding to the motion signal and a heart rate frequency corresponding to the target heart rate signal. As a further example, the processing device 110 may determine a target heart rate signal by processing, based on the motion signal and the second signal, the first signal.

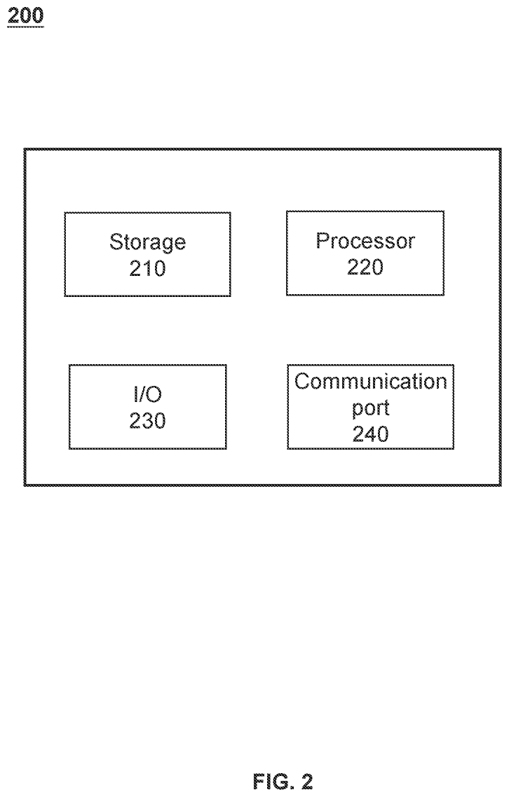

In some embodiments, the processing device 110 may be a single processing device or a processing device group, such as a server or a server group. The processing device group may be centralized or distributed (e.g., the processing device 110 may be a distributed system). In some embodiments, the processing device 110 may be local or remote. For example, the processing device 110 may access information and/or data in the collection device 120, terminal 130, and the storage device 140 through the network 150. As another example, the processing device 110 may be directly connected to the collection device 120, terminal 130, and the storage device 140 to access the information and/or data stored therein. In some embodiments, the processing device 110 may be implemented on a cloud platform. For example, the cloud platform may include a private cloud, a public cloud, a hybrid cloud, a community cloud, a distributed cloud, an inter-cloud, a multi-cloud, etc., or any combination thereof. In some embodiments, the processing device 110 may be implemented on a computing device shown in FIG. 2.

In some embodiments, the processing device 110 may include a processing engine 112. The processing engine 112 may process data and/or information related to the heart rate signal or motion signal to perform one or more processes or functions described in the present disclosure. For example, the processing device 112 may obtain a first signal of the user in the motion state and a motion signal corresponding to the motion signal. In some embodiments, the processing engine 112 may process the first signal and/or the motion signal to remove the motion signal and/or the second signal caused by the user's motion, thereby obtaining a target heart rate signal.

In some embodiments, the processing engine 112 may include one or more processing engines (e.g., a single-chip processing engine or a multi-chip processor). For example, the processing engine 112 may include a central processing unit (CPU), an application specific integrated circuit (ASIC), an application-specific instruction-set processor (ASIP), a graphics processing unit (GPU), a physics processing unit (PPU), a digital signal processor (DSP), a field programmable gate array (FPGA), a programmable logic device (PLD), a controller, a microcontroller unit, a reduced instruction set computer (RISC), a microprocessor, etc., or any combination thereof. In some embodiments, the processing engine 112 may be integrated into the collection device 120 or the terminal 130.

In some embodiments, the collection device 120 may be configured to collect a heart rate signal of the user and/or a motion signal representing a motion state of the user, such as the first signal and/or the motion signal. In some embodiments, the collection device 120 may be a signal collection device or a collection group including a plurality of collection devices (e.g., 120-1, . . . , 120-n). In some embodiments, the collection device 120 may be a device including one or more sensors (e.g., an acceleration sensor, a gyroscope, a heart rate sensor (e.g., a photoplethysmography (PPG) photoelectric sensor), etc.) or other signal collection components (e.g., smart bracelets, smart foot rings, smart collars, smart watches, smart gloves, etc.).

The collection device 1210 may convert the collected heart rate signal and/or the motion signal into electrical signals, and transmit the electrical signals to the processing device 110 for processing. In some embodiments, the heart rate signal collected by the collection device 120 may include an MA signal caused by the motion of the user. The processing device 110 may perform the noise reduction operation on the collected heart rate signal based on the heart rate signal and the motion signal collected by the collection device 120 to remove the interference of the user's motion, thereby obtaining a clean heart rate signal.

In some embodiments, information and/or data may be transmitted between the collection device 120 and the processing device 110, the terminal 130, and the storage device 140 through the network 150. In some embodiments, the collection device 120 may be directly connected to the processing device 110 or the storage device 140 to transmit the information and/or data. For example, the collection device 120 and the processing device 110 may be different parts in a same electronic device (e.g., a smart bracelet, a smart watch, etc.) and connected through metal wires.

In some embodiments, the terminal 130 may be a terminal used by the user or other entities. For example, the terminal 130 may be a terminal used to carry the collection device 120. For example, the terminal 130 may be a terminal used to communicate with any one or more components in the collection device 120 or the heart rate monitoring system 100 through the network 150. In some embodiments, the collection device 120 may be a part of the terminal device 130.

In some embodiments, the terminal 130 may include a mobile device 130-1, a tablet 130-2, a laptop 130-3, etc., or any combination thereof. In some embodiments, the mobile device 130-1 may include a smart home device, a wearable device, a smart mobile device, a virtual reality device, an augmented reality device, etc., or any combination thereof. In some embodiments, the smart home device may include a smart lighting device, a smart electrical control device, a smart monitoring device, a smart television, a smart camera, a walkie talkie, etc., or any combination thereof. In some embodiments, the wearable device may include a smart bracelet, a smart footwear, a pair of smart glasses, a smart helmet, a smart watch, a smart headphone, a smart clothing, a smart backpack, a smart accessory, etc., or any combination thereof. In some embodiments, the smart mobile device may include a smartphone, a personal digital assistant (PDA), a gaming device, a navigation device, a point of sale (POS), etc., or any combination thereof. In some embodiments, the virtual reality device and/or augmented reality device may include a virtual reality helmet, a pair of virtual reality glasses, a virtual reality goggle, an augmented virtual reality helmet, a pair of augmented reality glasses, an augmented reality goggle, etc., or any combination thereof.

In some embodiments, the terminal 130 may obtain/receive the heart rate signal and/or motion signal collected by the collection device 120. In some embodiments, the terminal 130 may obtain/receive a target heart rate signal obtained by the processing device 110 by processing the heart rate signal and/or motion signal. In some embodiments, the terminal 130 may directly obtain/receive signal or data from the collection device 120 and the storage device 140, such as a first signal including a superimposed signal between the heart rate signal and the motion signal and a motion signal representing the motion state of the user. In some embodiments, the terminal 130 may obtain/receive the clean heart rate signal obtained by performing the noise reduction operation from the storage device 140 or the processing device 110 through the network 150.

In some embodiments, the terminal 130 may send instructions to the processing device 110 and/or the collection device 120, and the processing device 110 and/or the collection device 120 may execute the instructions from the terminal 130. For example, the terminal 130 may send one or more instructions to the processing device 110 and/or the collection device 120 to implement the heart rate monitoring method such that the processing device 110 and/or the collection device 120 may perform one or more operations/steps of the heart rate monitoring method.

The storage device 140 may store data and/or information obtained from other devices or system components. In some embodiments, the storage device 140 may store data obtained from the collection device 120 or data processed by the processing device 110. For example, the storage device 140 may store the heart rate signal and/or the motion signal collected by the collection device 120 and store the target heart rate signal obtained by the processing device 110. In some embodiments, the storage device 140 may store data and/or instructions for the processing device 110 to execute or use to implement the exemplary methods described in the present disclosure. In some embodiments, the storage device 140 may include a mass memory, a removable memory, a volatile read-write memory, a read-only memory (ROM), etc., or any combination thereof. Exemplary mass memories may include disks, optical disks, solid-state disks, or the like. Exemplary removable memories may include flash drives, floppy disks, optical disks, storage cards, compressed disks, magnetic tapes, or the like. Exemplary volatile read-write memories may include a random access memory (RAM). Exemplary RAMs may include a dynamic RAM (DRAM), a double data rate synchronous dynamic RAM (DDR SDRAM), a static RAM (SRAM), a thyristor RAM (T-RAM), a zero capacitor RAM (Z-RAM), or the like. Exemplary ROMs may include a mask ROM (MROM), a programmable ROM (PROM), a programmable and erasable ROM (PEROM), an electrically erasable programmable ROM (EEPROM), a compact disc ROM (CD-ROM), a digital versatile disk ROM, or the like. In some embodiments, the storage device 140 may be implemented on a cloud platform. For example, the cloud platform may include a private cloud, a public cloud, a hybrid cloud, a community cloud, a distributed cloud, an internal cloud, a multi-layer cloud, etc., or any combination thereof.

In some embodiments, the storage device 140 may be connected with the network 150 to communicate with one or more components of the heart rate monitoring system 100 (e.g., the processing device 110, the collection device 120, the terminal 130). The components of the heart rate monitoring system 100 may access data or instructions stored in the storage device 140 through the network 150. In some embodiments, the storage device 140 may be directly connected to or communicated with the one or more components (e.g., the processing device 110, the collection device 120, the terminal 130) of the heart rate monitoring system 100. In some embodiments, the storage device 140 may be a part of the processing device 110.

In some embodiments, the one or more components of the heart rate monitoring system 100 (e.g., the processing device 110, the collection device 120, the terminal 130) may include a permission to access the storage device 140. In some embodiments, the one or more components of the heart rate monitoring system 100 may read and/or modify information related to the data when one or more conditions are satisfied.

The network 150 may facilitate the exchange of information and/or data. In some embodiments, the one or more components of the heart rate monitoring system 100 (e.g., the processing device 110, the collection device 120, the terminal 130, and the storage device 140) may send/receive information and/or data to/from other components of the heart rate monitoring system 100 through the network 150. For example, the processing device 110 may obtain the first signal and/or motion signal from the collection device 120 or the storage device 140 through the network 150, and the terminal 130 may obtain any one or more of the first signal, the motion signal, or the target heart rate signal from the processing device 110 or the storage device 140 through the network 150. In some embodiments, the network 150 may be any form of wired or wireless network, or any combination thereof. Merely by way of example, the network 150 may include a cable network, a wired network, an optical network, a telecommunication network, an internal network, an Internet, a local area network (LAN), a wide area network (WAN), a wireless local area network (WLAN), a metropolitan area network (MAN), a wide area network (WAN), a public switched telephone network (PSTN), a bluetooth network, a purple bee network, a near field communication (NFC) network, a global mobile communication system (GSM), a code division multiple access (CDMA) network, a time division multiple access (TDMA) network, a general packet radio service (GPRS) network, an enhanced data rate GSM evolution (EDGE) network, a wideband code division multiple access (WCDMA) network, a high speed downlink packet access (HSDPA) network, a long term evolution (LTE) network, a user datagram protocol (UDP) network, a transmission control protocol/internet protocol (TCP/IP) network, a short message service (SMS) network, a wireless application protocol (WAP) network, an ultra-wideband (UWB) network, an infrared ray, etc., or any combination thereof. In some embodiments, the system 100 may include one or more network access points. For example, the heart rate monitoring system 100 may include wired or wireless network access points, such as base stations and/or wireless access points 150-1, 150-2, . . . . The one or more components of the heart rate monitoring system 100 may be connected with the network 150 to exchange data and/or information.

Those skilled in the art may understand that when the components or elements of the heart rate monitoring system 100 operate, the components may operate through an electrical and/or electromagnetic signal. For example, when the collection device 120 sends the first signal and/or the motion signal to the processing device 110, the collection device 120 may generate an encoded electrical signal. Further, the collection device 120 may sent the electrical signal to an output port. If the collection device 120 communicates with the collection device 120 through a wired network or data transmission wire, the output port may be physically connected to a cable, and the cable may further transmit the electrical signal to an input port of the collection device 120. If the collection device 120 communicates with the collection device 120 through a wireless network, the output port of the collection device 120 may be one or more antennas that may convert the electrical signal into the electromagnetic signal. In an electronic device such as the collection device 120 and/or the processing device 110, when an instruction is processed, the instruction is issued, and/or an operation is performed, the instruction and/or operation may be in a form of the electrical signal. For example, when the processing device 110 reads data from or writes data on the storage medium (e.g., the storage device 140), the electrical signal may be sent to a read/write device of the storage medium such that structured data may be read from or write on the storage medium. The structured data may be transmitted to the processor in a form of an electrical signal through a bus of the electronic device. The electrical signal herein refers to an electrical signal, a series of electrical signals, and/or at least two discontinuous electrical signals.

FIG. 2 is a schematic diagram illustrating an exemplary computing device 200 according to some embodiments of the present disclosure. In some embodiments, the processing device 110 may be implemented on the computing device 200. As shown in FIG. 2, the computing device 200 may include a storage 210, a processor 220, an input/output (I/O) 230, and a communication port 240.

The storage 210 may store data/information obtained from the collection device 120, the terminal 130, the storage device 140, or any other components of the heart rate monitoring system 100. In some embodiments, the storage 210 may include a mass memory, a removable memory, a volatile read-write memory, a read-only memory (ROM), etc., or any combination thereof. Exemplary mass memories may include disks, optical disks, solid-state disks, or the like. Exemplary removable memories may include flash drives, floppy disks, optical disks, storage cards, compressed disks, magnetic tapes, or the like. Exemplary volatile read-write memories may include a random access memory (RAM). Exemplary RAMs may include a dynamic RAM (DRAM), a double data rate synchronous dynamic RAM (DDR SDRAM), a static RAM (SRAM), a thyristor RAM (T-RAM), and a zero capacitor RAM (Z-RAM). Exemplary ROMs may include a mask ROM (MROM), a programmable ROM (PROM), a programmable and erasable ROM (PEROM), an electrically erasable programmable ROM (EEPROM), a compact disc ROM (CD-ROM), a digital versatile disk ROM, or the like. In some embodiments, the storage 210 may store one or more programs and/or instructions to execute the exemplary methods described in the present disclosure. For example, the storage 210 may store programs that can be executed by the processing device 110 to implement a heart rate monitoring method.

The processor 220 may execute computer instructions (i.e., program codes) and the functions of the processing device 110 according to techniques described in the present disclosure. The computer instructions may include routines, programs, objects, components, signals, data structures, procedures, modules, and/or functions that perform specific functions described herein. For example, the processor 220 may process data obtained from the collection device 120, the terminal 130, the storage device 140, and/or any other components of the heart rate monitoring system 100. For example, the processor 220 may process the first signal and/or the motion signal obtained by the collection device 120 to remove the motion signal and/or the second signal caused by the user's motion, thereby obtaining the target heart rate signal. In some embodiments, the target heart rate signal obtained after a noise reduction operation may be stored in the storage device 140, the storage 210, or the like. In some embodiments, the target heart rate signal may be transmitted to output devices such as a display, a speaker, etc., through the I/O 230. In some embodiments, the processor 220 may perform instructions obtained from the terminal 130.

In some embodiments, the processor 220 may include one or more hardware processors, such as a microcontroller, a microprocessor, a reduced instruction set computer (RISC), an application specific integrated circuit (ASIC), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuits or processors capable of performing one or more functions, etc., or any combination thereof.

For illustrative purposes only, only one processor is described in the computing device 200. It should be noted that the computing device 200 in the present disclosure may also include multiple processors. Therefore, operations and/or operations executed by a processor as described in the present disclosure may also be jointly or separately executed by multiple processors. For example, in the present disclosure, the processor of the computing device 200 may perform operations A and B simultaneously, it should be understood that the operations A and B may also be executed jointly or separately by two or more different processors in the computing device. For example, a first processor may perform the operation A, and a second processor may perform the operation B, or the first and second processors may jointly perform operations A and B.

The I/O 230 may be configured to input or output signals, data, and/or information. In some embodiments, the I/O 230 may enable the user to interact with the processing device 110. In some embodiments, the I/O 230 may include an input device and an output device. Exemplary input devices may include a keyboard, a mouse, a touch screen, a microphone, etc., or any combination thereof. Exemplary output devices may include a display device, a speaker, a printer, a projector, etc., or combinations thereof. Exemplary display devices may include a liquid crystal display (LCD), a light-emitting diode (LED) based display, a display, a flat panel display, a curved screen, a television device, a cathode ray tube (CRT), a speaker, etc., or any combination thereof.

The communication port 240 may be connected with the network (e.g., the network 150) to facilitate data communication. The communication port 240 may establish a connection between the processing device 110 and the collection device 120, the terminal 130, or the storage device 140. The connection may be wired, wireless, or a combination thereof, to achieve data transmission and reception. The wired connection may include cables, optical cables, telephone lines, etc., or any combination thereof. The wireless connection may include Bluetooth, WiFi, WiMax, WLAN, ZigBee, mobile networks (e.g., 3G, 4G, 5G, etc.), etc., or a combination thereof. In some embodiments, the communication port 240 may be a standardized communication port, such as RS232, RS485, etc. In some embodiments, the communication port 240 may be a specially designed communication port. For example, the communication port 240 may be designed based on a signal that needs to be transmitted.

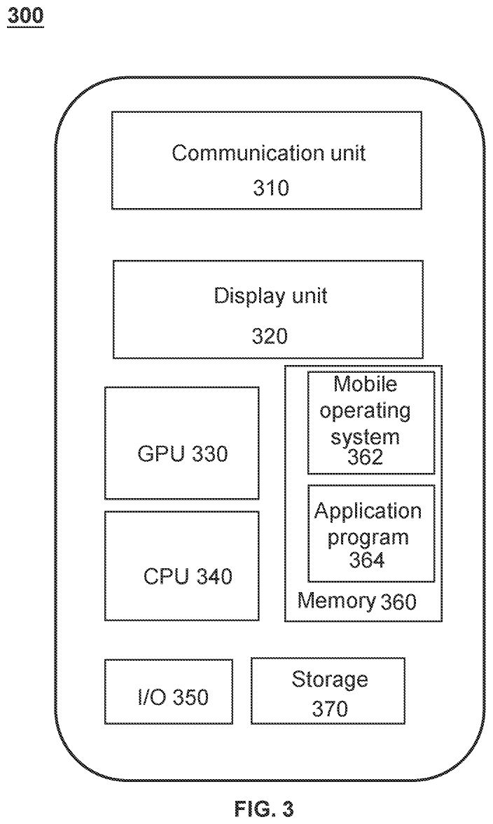

FIG. 3 is a schematic diagram illustrating exemplary hardware and/or software components of an exemplary mobile device 300 on which the terminal 130 may be implemented according to some embodiments of the present disclosure. As shown in FIG. 3, the mobile device 300 may include a communication unit 310, a display unit 320, a graphics processing unit (GPU) 330, a central processing unit (CPU) 340, an input/output 350, a memory 360, and a storage 370.

The CPU 340 may include an interface circuit and a processing circuit similar to the processor 220. In some embodiments, any other suitable components, including but not limited to system buses or controllers (not shown in the figures), may also be included in the mobile device 300. In some embodiments, a mobile operating system 362 (e.g., IOS™, Andro™, Windows Phone™, etc.) and one or more application programs 364 may be loaded from the storage 370 into the memory 360 and executed by the CPU 340. The one or more application programs 364 may include a browser or any other suitable mobile application programs for receiving, from a heart rate monitoring system on the mobile device 300, information related to the heart rate signal and presenting the information. The interaction of the signal and/or data may be implemented by the input/output device, and the signal and/or data may be provided to the processing engine 112 and/or other components of the heart rate monitoring system 100 through the network 150.

In order to implement the various modules, units, and functions mentioned above, a computer hardware platform may be used as a hardware platform for one or more elements (e.g., modules of the processing device 110 described in FIG. 1). Since the hardware components, operating systems, and programming languages are common, it may be assumed that those skilled in the art may be familiar with the technologies and may provide necessary information for heart rate monitoring based on the technologies described in the present disclosure. A computer with a user interface may be used as a personal computer (PC) or other types of workstations or the terminal device. After proper programming, the computer with the user interface may be used as a processing device such as a server. It should be considered that those skilled in the art may also be familiar with the structure, program, or general operation of the type of the computing device. Therefore, there is no additional explanation for the accompanying drawings.

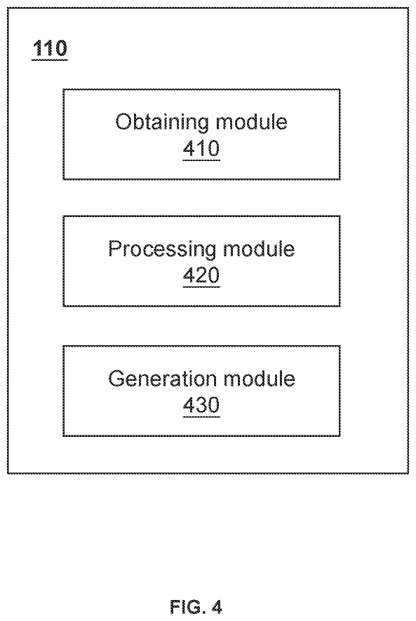

FIG. 4 is a block diagram illustrating an exemplary system for monitoring a heart rate according to some embodiments of the present disclosure. In some embodiments, the heart rate monitoring system 100 may be implemented on the processing device 110. As shown in FIG. 4, the processing device 110 may include an obtaining module 410, a processing module 420, and a generation module 430.

The obtaining module 410 may be configured to obtain a first signal. In some embodiments, the first signal may include a target heart rate signal in a motion state. In some embodiments, the first signal may include a motion signal corresponding to the motion state. In some embodiments, the first signal may include a superimposed signal between the motion signal and the target heart rate signal. In some embodiments, the first signal may be a heart rate signal collected by a collection device (e.g., the collection device 120) in the motion state of the user. In some embodiments, the collection device may collect the heart rate signal based on a photoplethysmographic (PPG) algorithm. The obtaining module 410 may obtain the first signal from the collection device. In some embodiments, the first signal may be stored in a storage device (e.g., the storage device 140, the storage 220, the storage 370, or external storage devices). The obtaining module 410 may obtain the first signal from the storage device.

The processing module 420 may be configured to obtain a motion signal corresponding to the motion state. In some embodiments, to obtain the motion signal corresponding to the motion state, the processing module 420 may be configured to obtain a filtered signal by performing a filtering operation on the first signal to reduce or filter out a noise signal (e.g., a baseline drift, etc.) in the first signal. For example, the processing module 420 may perform the filtering operation on the first signal based on a filtering algorithm to reduce or filter out the baseline drift. Further, the processing module 420 may determine the motion signal corresponding to the motion state based on the filtered signal. For example, the processing module 420 may determine the motion signal corresponding to the motion state by processing, based on an independent component analysis (ICA) algorithm such that the filtered signal may be statistically independent. In such cases, independent components corresponding to the target heart rate signal and motion signal, respectively may be obtained. As another example, the processing module 420 may designate a signal with a specific frequency component in the filtered signal as the motion signal.

In some embodiments, to obtain the motion signal corresponding to the motion state, the processing module 420 may be configured to determine the motion signal based on a motion collection device (e.g., an acceleration sensor, a gyroscope, a magnetometer, etc.).

In some embodiments, to obtain the motion signal corresponding to the motion state, the processing module 420 may be configured to obtain two or more first signals through two or more optical paths. For example, the processing module 420 may cause the two or more optical paths to emit lights with two or more spectral distributions (e.g., having two or more different wavelengths). The collection device may obtain the two or more first signals corresponding to the lights with two or more spectral distributions, respectively. In some embodiments, the lights with the two or more different spectral distributions may have a same or similar correlation with the motion signal. Correspondingly, the two or more first signals may have a common-mode signal. The common-mode signal may correspond to the motion signal. In some embodiments, the lights with two or more different spectral distributions may have different correlations with the target heart rate signal. Correspondingly, the two or more first signals may have a differential signal. The differential signal may correspond to the target heart rate signal. Further, the processing module 420 may be configured to determine the motion signal based on the two or more first signals. For example, the processing module 420 may obtain the common-mode signal by separating the common-mode signal from the differential signal. The common-mode signal may be designated as the motion signal.

The processing module 420 may be configured to identify a second signal with a target frequency from the first signal based on a motion frequency corresponding to the motion signal and a heart rate frequency corresponding to the target heart rate signal. In some embodiments, after determining the motion signal, the processing module 420 may be configured to determine the motion frequency corresponding to the motion signal. In some embodiments, the processing module 420 may be configured to obtain a preliminary target heart rate signal by removing the motion signal from the first signal based on a filtering operation. Further, the processing module 420 may be configured to determine a heart rate frequency corresponding to the preliminary target heart rate signal and designate the heart rate frequency corresponding to the preliminary target heart rate signal as the heart rate frequency corresponding to the target heart rate signal. In some embodiments, the processing module 420 may be configured to convert the first signal into a frequency domain signal through a Fast Fourier Transform (FFT) operation. Further, the processing module 420 may determine the motion frequency corresponding to the motion signal and the heart rate frequency corresponding to the target heart rate signal based on the frequency domain signal. In some embodiments, the second signal may correspond to a non-linear superimposed signal between the target heart rate signal and the motion signal. The second signal may have a target frequency. The target frequency may originate from a linear superposition of the motion frequency and the heart rate frequency corresponding to the target heart rate signal. In some embodiments, the target frequency corresponding to the second signal may be equal to a sum of the motion frequency corresponding to the motion signal and the heart rate frequency corresponding to the target heart rate frequency. In some embodiments, the target frequency corresponding to the second signal may be equal to a difference between the motion frequency corresponding to the motion signal and the heart rate frequency corresponding to the target heart rate signal. In some embodiments, the sum or difference between the motion frequency and the heart rate frequency may include the sum or difference between a multiple of the motion frequency and a multiple of the heart rate frequency. In such cases, the processing module 420 may determine the target frequency based on the motion frequency corresponding to the motion signal and the heart rate frequency corresponding to the target heart rate signal. Further, the processing module 420 may identify the second signal from the first signal based on the target frequency.

The generation module 430 may be configured to determine the target heart rate signal by processing, based on the motion signal and the second signal, the first signal. In some embodiments, to determine the target heart rate signal, the generation module 430 may be configured to determine the target heart rate signal by removing the motion signal and/or the second signal from the first signal. In some embodiments, the generation module 430 may remove the motion signal by performing the filtering operation on the first signal after determining the motion signal. In some embodiments, the generation module 430 may determine the target heart rate signal by deleting the second signal corresponding to the target frequency directly. Alternatively or additionally, the generation module 430 may further perform a smoothing operation on the first signal after deleting the second signal, thereby determining the target heart rate signal. In some embodiments, to determine the target heart rate signal, the generation module 430 may replace the second signal corresponding to the target frequency with a reference heart rate signal. The reference heart rate signal may be a predetermined signal or signal range determined based on heart rate signal statistical data. In some embodiments, to determine the target heart rate signal, the generation module 430 may further determine a motion component and/or a heart rate component in the second signal, and process the second signal based on the motion component and/or the heart rate component. The motion component and the heart rate component may respectively refer to an influence degree of the motion signal and the target heart rate signal on the second signal, which may be determined based on data analysis or in other manners.

It should be understood that the system and modules shown in FIG. 4 can be implemented in various ways. For example, in some embodiments, the system and its modules may be implemented through hardware, software, or a combination of software and hardware. The hardware part may be implemented using dedicated logic; the software part may be stored in the storage and executed by an appropriate instruction execution system, such as a microprocessor or specialized design hardware. Those skilled in the art may understand that the above methods and systems may be implemented using computer executable instructions and/or included in processor control code, such as providing such code on carrier media such as disks, CDs, or DVD-ROMs, programmable memory such as read-only memory (e.g., a firmware), or data carriers such as optical or electronic signal carriers. The system and modules described in the present disclosure may be implemented not only by hardware circuits such as ultra large scale integrated circuits or gate arrays, semiconductors such as logic chips, transistors, or programmable hardware devices such as field programmable gate arrays and programmable logic devices, but also by software executed by various types of processors, or by combining the hardware circuit and software mentioned above (e.g., a firmware).

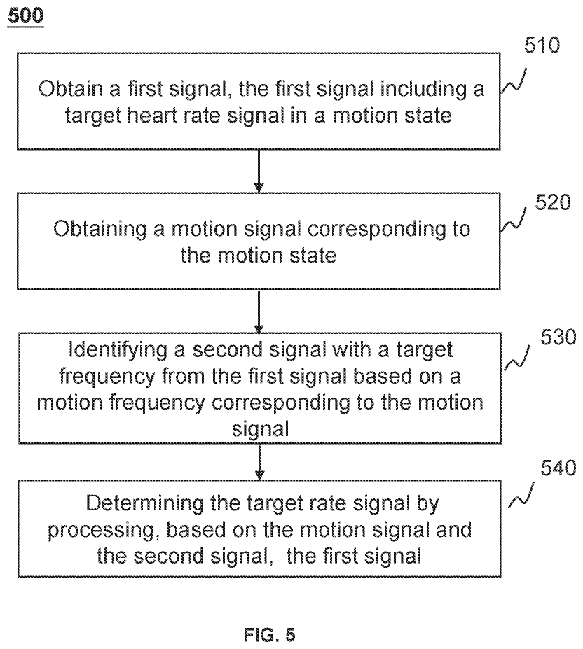

FIG. 5 is a flowchart illustrating an exemplary process for monitoring a heart rate according to some embodiments of the present disclosure. In some embodiments, the process 500 may be performed by the processing device 110, the processing engine 112, or the processor 220. For example, the process 500 may be stored in a storage device (e.g., the storage device 140 or a storage unit of the processing device 110) in a form of programs or instructions, when the processing device 110, the processing engine 112, the processor 220, or the modules shown in FIG. 4 perform the instructions or programs, the process 500 may be implemented. In some embodiments, the process 500 may be implemented according to one or more additional operations not described below, and/or without one or more operations described below. In addition, the sequence of operations shown in FIG. 5 is not limiting. As shown in FIG. 5, the process 500 may include:

In 510, the processing device 110 (e.g., the obtaining module 410) may obtain a first signal.

In some embodiments, the first signal may include a target heart rate signal in a motion state. The target heart rate signal refers to a heart rate signal (i.e., a clean heart rate signal) without a noise signal (e.g., motion artifacts (MA), etc.). In some embodiments, the first signal may include a motion signal corresponding to the motion state. The motion signal may generate an interference during a signal collection process, causing changes in a waveform of the heart rate signal collected by the collection device, thereby forming the MA. In some embodiments, the motion signal also refers to a MA signal. In some embodiments, the first signal may further include a superimposed signal between the motion signal and the target heart rate signal. In some embodiments, the superimposed signal may include a non-linear superimposed signal.

In some embodiments, the first signal may be a heart rate signal collected by a collection device (e.g., the collection device 120) in the motion state of the user. The collection device may include a heart rate sensor. Exemplary sensors may include a photoelectric secondary sensor, a complementary metal oxide semiconductor sensor, or the like. In some embodiments, the processing device 110 may obtain the first signal from the collection device. In some embodiments, the first signal may be stored in a storage device (e.g., the storage device 140, the storage 220, the storage 370, or an external storage device). The processing device 110 may obtain the first signal from the storage device.

In some embodiments, the collection device may collect the heart rate signal based on a photoplethysmographic (PPG) algorithm. The PPG algorithm may utilize a principle that a photoelectric sensing component can absorb light energy. According to the PPG algorithm, light (e.g., a LED light) may be used to irradiate a skin of an object. Further, changes of the light in blood vessels due to the blood flow may be recorded by the photoelectric sensing component such that the heart rate signal may be obtained.

Taking a collection of the first signal based on the PPG algorithm as an example, in some embodiments, the propagation of light in substances may follow a Lambert-Beer law:

T=e^{-Σ}^{ᵢ₌₁}^{^{N}}^{σ}^{ᵢ}^{∫}^{₀}^{^{l}}ⁿ^{ᵢ}^{(z)dz}, (1)

wherein T denotes a transmissivity, N denotes N kinds of substances, σᵢ denotes a loss cross-section of an ith substance, nᵢ denotes a density of the ith substance, and l denotes an optical path.

It should be noted that Equation (1) shows a derivation of transmission of the light in substances. In some embodiments, a reflection of the light may also be analyzed referring to the transmission. In some embodiments, a tissue (e.g., a wrist used for measuring a heart rate) may include an artery (A), a vein (V), and other tissues (T, such as a bone, a muscle tissue, etc., assuming that there is no blood in the other tissues). The A, V, and T may be used to denote optical paths of the light in the three types of tissues. Assuming that the densities of the three types of tissues are uniform and only the optical path is considered, a transmitted light intensity received by the photoelectric sensing component without the influence of the heart rate and the motion may be:

lₜ=l₀*e^{-(α}^{_{T}}^{T+α}^{_{V}}^{V+α}^{_{A}}^{A)}, (2)

wherein lₜ denotes the transmitted light intensity received by the photoelectric sensing component, l₀ denotes an incident light intensity, and α denotes an absorption coefficient.

In some embodiments, the heart rate and motion may cause changes of the shape and position of arterial blood vessels, resulting in changes of the optical path l in Equation (1). The heart rate and motion may also cause changes of a blood flow density, resulting in changes of the absorption coefficient α in Equation (2). In such cases, a change of the optical path caused by the heart rate may be ΔA, and a change of the absorption coefficient caused by the heart rate may be Δα_{A}. The motion has a similar influence on the artery and the vein. In such cases, changes of the optical paths in the artery and the vein caused by the motion may be Δα_{Am} and Δαᵥₘ, respectively, and changes of the absorption coefficients of the artery and the vein caused by the motion may be Δα_{Am} and Δαᵥₘ, respectively. In such cases, the transmitted light intensity received by the photoelectric sensing component may be:

lₘ=l₀*e^{-(α}^{_{T}}^{T+(α}^{ᵥ}^{+Δα}^{ᵥₘ}^{)(V+ΔV}^{ₘ}^{)+(α}^{_{A}}^{+Δα}^{_{Am}}^{+Δα}^{_{A}}^{)(A+ΔA}^{ₘ}^{+ΔA))}, (3)

wherein lₘ denotes a transmitted light intensity received by the photoelectric sensing component in the motion state. A transmitted light signal corresponding to the transmitted light intensity may be converted into an electrical signal, which may include a heart rate signal in the motion state (i.e., the first signal).

In some embodiments, since the absorption of light by the artery changes in the motion state while the absorption of the light by other tissues is basically unchanged, the transmitted light signal may include a direct current (DC) signal and an alternation current (AC) signal, wherein the DC signal may be used to detect reflected light signals of tissues, bones, and muscles, and the AC signal denotes a change of blood volume generated between a systolic phase and a diastolic phase of a cardiac cycle. Therefore, when converting the transmitted light signal received by the photoelectric sensing component into an electrical signal, the AC signal may be extracted. The AC signal reflects characteristics of blood flow. In such cases, the heart rate signal in the motion state may be estimated based on the AC signal. Thus the first signal may be represented as the AC signal.

In some embodiments, according to Equation (3), the first signal may include a superimposed signal Δα_{A}ΔAₘ (i.e., the second signal) between the motion signal and the target heart rate signal. The superimposed signal between the motion signal and the target heart rate signal herein refers to a noise signal generated by an interaction between the motion signal and the target heart rate signal, which is related to amplitudes and frequencies of the motion signal and the target heart rate signal.

In 520, the processing device 110 (e.g., the processing module 420) may obtain a motion signal corresponding to the motion state. The motion signal may represent a motion state of the user. In some embodiments, the motion signal may at least include a motion frequency corresponding to the motion state.

In some embodiments, the first signal may include a noise signal. For example, the noise signal may include an environmental noise, a baseline drift, or the like. The environmental noise refers to an interference generated by environment signals (e.g., an electromagnetic signal, or an ambient light signal). In some embodiments, a shielding component may be disposed on the collection device to shield the interference of the environmental signal. The baseline drift refers to a slow change of the baseline in the first signal with the temporal orientation. The baseline drift may be caused by a breathing fluctuation of the human body during a measurement process and/or a relative friction between a skin surface and the collection device. In some embodiments, the baseline drift may be a low-frequency noise. Merely by way of example, the frequency of the baseline drift may be in a range of 0 Hz-1 Hz.

In some embodiments, to obtain the motion signal corresponding to the motion state, the processing device 110 may obtain a filtered signal by performing a filtering operation on the first signal to reduce or remove the noise signal in the first signal. For example, the processing device 110 may perform the filtering operation on the first signal based on a filtering algorithm to reduce or filter out the baseline drift. Exemplary filtering algorithms may include a finite impulse response (FIR) filtering algorithm, an adaptive median filtering algorithm, an infinite impulse response (IIR) filtering algorithm, or the like. Merely by way of example, the processing device 110 may perform a high pass filtering operation on the first signal based on the filtering algorithm to reduce or filter out the baseline drift. In some embodiments, a cutoff frequency of the high pass filtering operation may be determined based on a frequency of the baseline drift. For example, if the frequency of the baseline drift is in a range of 0 Hz-1 Hz, the cutoff frequency of the high pass filtering operation may be 1 Hz. After a filtering operation is performed on the first signal based on the cutoff frequency, the baseline drift below 1 Hz may be reduced or filtered out. The filtered signal may include the motion signal and the target heart rate signal. Further, the processing device 110 may determine the motion signal corresponding to the motion state based on the filtered signal. For example, the processing device 110 may determine the motion signal by processing the filtered signal based on an independent component analysis (ICA) algorithm. The ICA algorithm may separate data or signal (e.g., the filtered signal) into statistical and non-Gaussian independent components based on a statistical principle. The processing device 110 may obtain the independent components corresponding to the target heart rate signal and the motion signal, respectively by making, based on the ICA algorithm, the filtered signal statistically independent, thereby determining the motion signal corresponding to the motion state. As another example, the processing device 110 may designate a signal with a specific frequency component in the filtered signal as the motion signal. Merely by way of example, the processing device 110 may extract a signal within the specific frequency range (e.g., 3 Hz-5 Hz, 3 Hz-8 Hz) from the filtered signal and identify the motion signal based on the signal within the specific frequency range. In some embodiments, the specific frequency range may be determined based on a reference heart rate frequency of the user. Merely by way of example, the reference heart rate frequency of the user may be directly determined by the system or extracted from historical heart rate data of the user or other users. The reference heart rate frequency of the user may be outside the specific frequency range. Optionally, the processing device 110 may unify the signal within the specific frequency range as the motion signal, or further extract a signal component (e.g., one or more frequency components corresponding to a maximum amplitude) with specific features within the frequency range as the motion signal.

In some embodiments, to obtain the motion signal corresponding to the motion state, the processing device 110 may determine the motion signal based on a motion collection device. The motion collection device may be integrated into the collection device for collecting the first signal, or may be used as an independent device for collecting the motion signal. Merely by way of example, the motion collection device may include an acceleration sensor, a gyroscope, a magnetometer, or the like. The processing device 110 may obtain parameters such as an acceleration, an angular velocity, etc. of the user in the motion state through the acceleration sensor, gyroscope, magnetometer, or the like, and process the parameters through a data fusion algorithm to determine the motion signal. In some embodiments, by determining the motion signal through the motion collection device, a relatively more accurate motion signal may be obtained, and a corresponding processing of the first signal may be avoided, thereby improving the accuracy and acquisition efficiency of the motion signal.

In some embodiments, to obtain the motion signal corresponding to the motion state, the processing device 110 may obtain two or more first signals through two or more optical paths. In some embodiments, the collection device may have two or more optical paths, and the processing device 110 may cause the two or more optical paths to emit lights with two or more spectral distributions. The two or more spectral distributions may include two or more different wavelengths. Taking that the collection device includes a first optical path and a second optical path as an example, the first and second optical paths may emit lights with different wavelengths, respectively. For example, the first optical path may emit light with a relatively shorter wavelength (e.g., a green light), and the second optical path may emit light with a relatively longer wavelength (e.g., a red light). In some embodiments, the two or more different wavelengths of light may alternately irradiate into the skin of the user. The collection device may separately obtain two or more first signals corresponding to the lights with different wavelengths. In some embodiments, the lights with two or more different wavelengths may have the same or similar correlation with the motion signal. Correspondingly, the two or more first signals may have a common-mode signal (i.e., a common part of the two or more first signals). The common-mode signal may correspond to the motion signal. In some embodiments, the lights with two or more different wavelengths may have different correlations with the target heart rate signal. Correspondingly, the two or more first signals may include a differential signal (i.e., a different part between the two or more first signals). The differential signal may correspond to the target heart rate signal. Further, the processing device 110 may determine the motion signal based on the two or more first signals. For example, the processing device 110 may obtain the common-mode signal by separating the common-mode signal and the differential signal. The common-mode signal may be designated as the motion signal.

In 520, the processing device 110 (e.g., the processing module 420) may identify a second signal with a target frequency from the first signal based on a motion frequency corresponding to the motion signal.

In some embodiments, the processing device 110 may identify the second signal with the target frequency from the first signal based on the motion frequency and a heart rate frequency corresponding to the target heart rate signal. In some embodiments, after determining the motion signal, the processing device 110 may further determine the motion frequency corresponding to the motion signal. In some embodiments, the processing device 110 may obtain a preliminary target heart rate signal by removing the motion signal from the first signal through the filtering operation. The preliminary target heart rate signal may be used as a target heart rate signal obtained based on a rough calculation, which may include a superimposed signal between the motion signal and the target heart rate signal. The processing device 110 may determine the heart rate frequency corresponding to the preliminary target heart rate signal and designate the heart rate frequency corresponding to the preliminary target heart rate signal as the heart rate frequency corresponding to the target heart rate signal. In some embodiments, the processing device 110 may convert the first signal into a frequency domain signal through a Fast Fourier Transform (FFT) operation, and determine the motion frequency corresponding to the motion signal and the heart rate frequency corresponding to the target heart rate signal based on the frequency domain signal. For example, when the motion frequency and the heart rate frequency is determined, the first signal may be approximately considered as a linear superposition of the target heart rate signal and the motion signal. The first signal may be decomposed into waveform components with the motion frequency and the heart rate frequency, respectively through the FFT operation. In such cases, the processing device 110 may determine the motion frequency and the heart rate frequency based on a result of the FFT operation.

In some embodiments, according to Equation (3), the first signal may include the target heart rate signal, the motion signal, and the superimposed signal Δα_{A}ΔAₘ of the target heart rate signal and the motion signal. The superimposed signal may be a non-linear multiplicative superimposed signal. In some embodiments, according to a product-to-sum law, the non-linear superimposed signal may be converted into a linear superimposed signal. The converted linear superimposed signal has a new signal frequency. The new signal frequency may be related to the motion frequency and the heart rate frequency corresponding to the target heart rate signal, wherein the converted linear superimposed signal is the second signal. The second signal has a target frequency, i.e., the new signal frequency. In some embodiments, the target frequency corresponding to the second signal may be equal to a sum of the motion frequency corresponding to the motion signal and the heart rate frequency corresponding to the target heart rate signal. In some embodiments, the target frequency corresponding to the second signal may be equal to a difference between the motion frequency corresponding to the motion signal and the heart rate frequency corresponding to the target heart rate signal. In some embodiments, the sum or difference of the motion frequency and the heart rate frequency may include a sum or difference between a multiple of the motion frequency and a multiple of the heart rate frequency. In such cases, the processing device 110 may determine the target frequency based on the motion frequency corresponding to the motion signal and the heart rate frequency corresponding to the target heart rate signal. Further, the processing device 110 may identify the second signal from the first signal based on the target frequency.

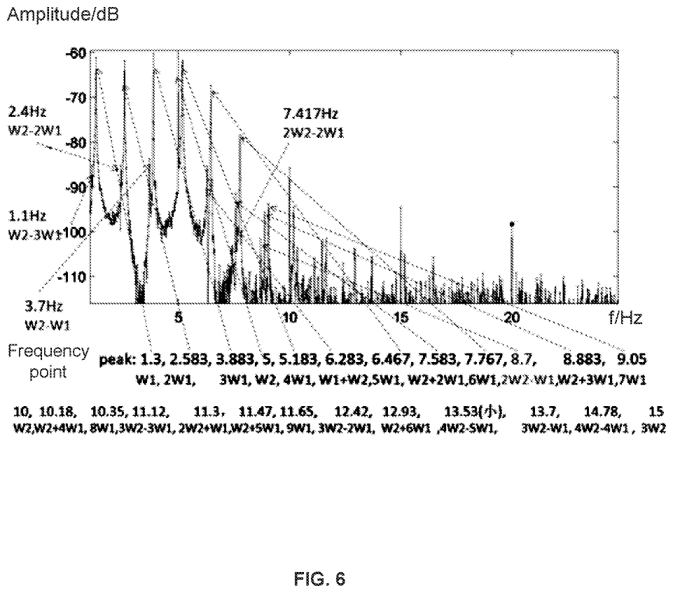

FIG. 6 is a schematic diagram illustrating an exemplary spectrum of a first signal according to some embodiments of the present disclosure. For illustration purposes, the first signal may be a signal obtained through an experimental simulation of a motion and a heart rate, wherein W1 denotes a heart rate frequency corresponding to the heart rate signal, and W2 denotes a motion frequency corresponding to the motion signal. During the experimental simulation process, the heart rate frequency and the motion frequency may be known parameters, wherein W1=1.3 Hz, and W2=5 Hz. After the first signal is obtained, the first signal may be converted into a frequency domain signal as shown in FIG. 6 through the FFT operation. As shown in FIG. 6, the horizontal axis denotes a frequency of the first signal, and the vertical axis denotes an amplitude of the first signal (e.g., an amplitude obtained according to a logarithmic calculation). In some embodiments, according to the FFT operation, there may be multiplied signals in the first signal converted to the frequency domain, i.e., signals with frequency points located at M*W1 and N*W2 (M=1, 2, 3, 4, 5, 6, 7, 8 . . . ; N=1, 2, 3, 4, 5, 6, 7, 8 . . . ). A position of each frequency peak in the first signal is shown in FIG. 6. As shown in FIG. 6, in addition to having multiplied signals at multiplied points with frequency points located at such as 2W1 (2.583 Hz), 3W1 (3.883 Hz), 2W2 (10 Hz), 3W2 (15 Hz), etc., the first signal also has frequency peaks with frequency points located at abs(W1±W2) (e.g., W1+W2, W2−W1, 2W1+W2, W2−2W1, etc.). In such cases, according to FIG. 6, the first signal may further include a non-linear superimposed signal (i.e., the second signal) between the motion signal and the target heart rate signal. The non-linear superimposed signal has a frequency peak(s) at the frequency point of abs(W1±W2). In such cases, the processing device 110 may determine the target frequency based on the motion frequency corresponding to the motion signal and the heart rate frequency corresponding to the target heart rate signal, and identify the second signal from the first signal based on the target frequency.

In some embodiments, the heart rate frequency corresponding to the target heart rate signal may be an unknown frequency, and the processing device 110 may identify the second signal with the target frequency from the first signal based on the motion frequency corresponding to the motion signal. For example, the unknown frequency may be X, and the motion frequency may be W2, according to the linear superposition relationship between the motion frequency and the heart rate frequency, the second signal has a frequency peak(s) at the frequency point(s) (i.e., the target frequency) of abs (X±W2). Therefore, the processing device 110 may identify the second signal with the target frequency of abs(X±W2) from the first signal based on the motion frequency according to the linear superposition relationship between the motion frequency and the heart rate frequency. In some embodiments, the processing device 110 may determine a heart rate frequency X based on the motion frequency according to the linear superposition relationship between the motion frequency and the heart rate frequency.

In 540, the processing device 110 (e.g., the generation module 430) may determine the target heart rate signal by processing, based on the motion signal and the second signal, the first signal.

In some embodiments, the processing device 110 may determine the target heart rate signal by removing the motion signal and/or the second signal from the first signal. In some embodiments, after determining the motion signal, the processing device 110 may perform a filtering operation on the first signal to remove the motion signal. In some embodiments, the processing device 110 may directly delete the second signal corresponding to the target frequency to determine the target heart rate signal. Alternatively or additionally, after deleting the second signal, the processing device 110 may perform a smoothing operation on the first signal to determine the target heart rate signal.

In some embodiments, to determine the target heart rate signal, the processing device 110 may replace the second signal corresponding to the target frequency with a reference heart rate signal. For example, the reference heart rate signal may be a predetermined signal or signal range based on the heart rate signal statistical data. Different motion frequencies may correspond to different reference heart rate signals. After determining the motion frequency, the processing device 110 may determine the reference heart rate signal corresponding to the motion frequency. Further, the processing device 110 may replace the second signal with the reference heart rate signal to determine the target heart rate signal.

In some embodiments, to determine the target heart rate signal, the processing device 110 may determine a motion component and/or a heart rate component in the second signal and process the second signal based on the motion component and/or the heart rate component. The motion component and the heart rate component respectively refer to influence degrees of the motion signal and the target heart rate signal on the second signal. Merely by way of example, first signals of a same object in different motion states and/or first signals of different objects in a same motion state may be collected and/or simulated, and the second signal in each first signal may be identified, respectively. Further, a relationship between the motion signal and the second signal may be determined based on a data analysis algorithm (e.g., a mathematical statistics algorithm, a machine learning algorithm, etc.). For example, the second signals corresponding to different motion signals may be analyzed based on the data analysis algorithm such that a variation law of the second signal with the motion signal may be determined. The variation law reflects an influence of the motion signal on the motion components in the second signal. Merely by way of example, the variation law may include a mapping relationship between a proportion of the motion components in the motion signal and the second signal. The processing device 110 may determine the motion components in the second signal based on the mapping relationship.

It should be noted that the process 500 for heart rate monitoring is merely provided for the purposes of illustration, and not intended to limit the scope of the present disclosure. For those skilled in the art, multiple variations and modifications for the imaging system and/or the detector may be made under the teachings of the present disclosure. For example, when a motion amplitude or motion frequency of the user is relatively small, an influence of the motion state of the user on the heart rate may also be relatively small. In such cases, it may be determined whether to accurately calculate the target heart rate signal based on the motion state of the user. Therefore, the process 500 may further include an operation of analyzing the motion state. As another example, when the heart rate frequency is an unknown frequency, the processing device 110 may identify the second signal with a target frequency of abs(X±W2) from the first signal based on the motion frequency according to the linear superposition relationship between the motion frequency and the heart rate frequency and determine the heart rate frequency X. In such cases, the operation 540 may be omitted, and the processing device 110 may identify the target heart rate signal from the first signal based on the heart rate frequency.

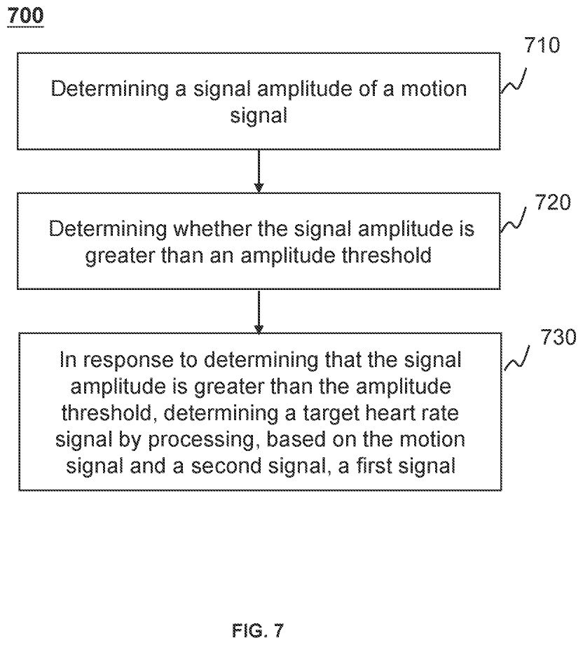

FIG. 7 is a flowchart illustrating an exemplary process for monitoring a heart rate according to some embodiments of the present disclosure. In some embodiments, the process 700 may be performed by the processing device 110, the processing engine 112, and the processor 220. For example, the process 700 may be stored in the form of programs or instructions in a storage device (e.g., a storage unit of the storage device 140 or the processing device 110). When the processing device 110, the processing engine 112, the processor 220, or the modules shown in FIG. 4 executes the programs or instructions, the process 700 may be implemented. In some embodiments, operation 520 described in the process 500 may be implemented through the process 700. In some embodiments, the process 700 may be implemented according to one or more additional operations not described below, and/or without one or more operations described below. In addition, the sequence of operations shown in FIG. 7 is not limiting. As shown in FIG. 7, the process 700 may include:

In 710, the processing device 110 (e.g., the processing module 420) may determine a signal amplitude of the motion signal. In some embodiments, the processing device 110 may determine the motion signal by performing the operation 510 and/or the operation 520 described in FIG. 5, which may not be repeated here. Further, the processing device 110 may determine the signal amplitude of the motion signal.

In 720, the processing device 110 (e.g., the processing module 420) may determine whether the signal amplitude of the motion signal is greater than an amplitude threshold. In some embodiments, the amplitude threshold may be a predetermined amplitude threshold based on historical heart rate data. For example, influences of the motions with different signal amplitudes on the target heart rate signal may be determined based on the historical heart rate data, and the signal amplitude corresponding to a relatively greater degree of influence may be determined as the amplitude threshold.

In 730, in response to determining that the signal amplitude is greater than the amplitude threshold, the processing device 110 (e.g., the generation module 430) may determine the target heart rate signal by processing, based on the motion signal and the second signal, the first signal. In some embodiments, the processing device 110 may determine the target heart rate signal by performing the operation 540 described in FIG. 5, which is not repeated here.

As shown in FIG. 7, whether to perform the operation 540 to determine an accurate target heart rate signal may be determined by determining whether the signal amplitude of the motion signal is greater than the amplitude threshold. In response to determining that the signal amplitude is greater than the amplitude threshold, the operation 540 may be performed to perform an accurate heart rate calculation to obtain the target heart rate signal of the user in the motion state; and in response to determining that the signal amplitude is equal to or less than the amplitude threshold, the heart rate signal corresponding to the first signal may be designated as the target heart rate signal, thereby reducing a computational load of the processor and improving a calculation speed of the heart rate monitoring while ensuring the accuracy of heart rate monitoring.

It should be noted that the process 700 for heart rate monitoring is merely provided for the purposes of illustration, and not intended to limit the scope of the present disclosure. For persons having ordinary skills in the art, multiple variations and modifications for the imaging system and/or the detector may be made under the teachings of the present disclosure.

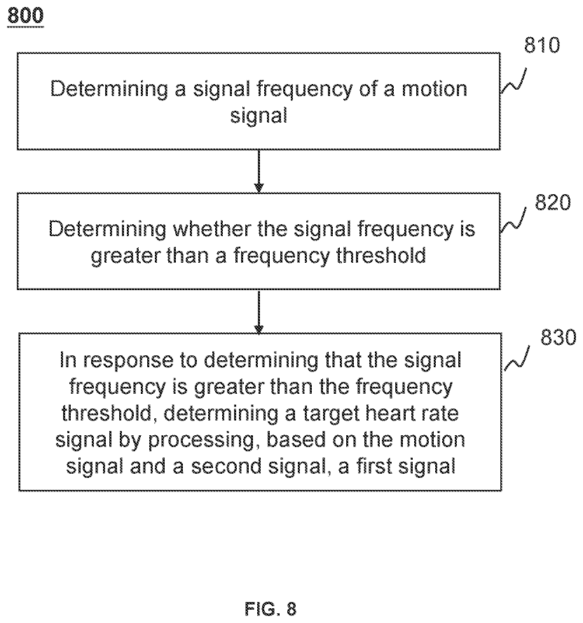

FIG. 8 is a flowchart illustrating an exemplary process for monitoring a heart rate according to other embodiments of the present disclosure. In some embodiments, the process 800 may be performed by the processing device 110, the processing engine 112, and the processor 220. For example, the process 800 may be stored in the form of programs or instructions in a storage device (e.g., a storage unit of storage device 140 or the processing device 110), and the process 800 may be implemented when the processing device 110, the processing engine 112, the processor 220, or the module shown in FIG. 4 executes the programs or instructions. In some embodiments, the operation 520 described in the process 500 may be implemented through the process 800. In some embodiments, the process 800 may be implemented according to one or more additional operations not described below, and/or without one or more of the operations described below. In addition, the sequence of operations shown in FIG. 8 is not limiting. As shown in FIG. 8, the process 800 may include:

In 810, the processing device 110 (e.g., the processing module 420) may determine a signal frequency (i.e., the motion frequency) of the motion signal. In some embodiments, the processing device 110 may determine the motion signal by performing the operation 510 and/or the operation 520 described in FIG. 7, which may not be further described here. Furthermore, the processing device 110 may determine the signal frequency of the motion signal.

In 820, the processing device 110 (e.g., the processing module 420) may determine whether the signal frequency is greater than the frequency threshold. In some embodiments, the frequency threshold may be a predetermined frequency threshold based on the historical heart rate data. For example, influences of the motions with different frequencies on the target heart rate signal may be determined based on the historical heart rate data, and the signal frequency corresponding to a relatively greater degree of influence may be determined as the frequency threshold.

In 830, in response to determining that the signal frequency is greater than the frequency threshold, the processing device 110 (e.g., the generation module 430) may determine the target heart rate signal by processing, based on the motion signal and the second signal, the first signal.

Similar to the process 700, in other embodiments, whether to perform the operation 540 may be determined by determining whether the signal frequency corresponding to the motion signal is greater than the frequency threshold. Specifically, in response to determining that the signal frequency of the motion signal is greater than the frequency threshold, the operation 540 may be performed o perform an accurate heart rate calculation to obtain the target heart rate signal of the user in the motion state; and in response to determining that the signal frequency of the motion signal is less than or equal to the frequency threshold, the heart rate signal corresponding to the first signal may be directly designated as the target heart rate signal. In some embodiments, the processing device 110 may determine the target heart rate signal by performing the operation 540 described in FIG. 5, which is not repeated here.

It should be noted that the process 800 for heart rate monitoring is merely provided for the purposes of illustration, and not intended to limit the scope of the present disclosure. For persons having ordinary skills in the art, multiple variations and modifications for the imaging system and/or the detector may be made under the teachings of the present disclosure.

The beneficial effects in the embodiments of the present discourse may include but are not limited to: (1) the method for monitoring a heart rate provided in the present disclosure can better remove the motion artifacts and the impact of motion noise on the heart rate signal by performing, based on the superposition relationship between the motion signal and the heart rate signal, a noise reduction operation on the data monitored by the heart rate sensor, thereby obtaining more accurate heart rate monitoring result; (2) the method for monitoring a heart rate provided in the present disclosure can accurately or roughly calculate the data monitored by the heart rate sensor based on the motion amplitude or motion frequency of the user, which can reduce the computational load of the processor when the motion amplitude of the user is relatively small, thereby ensuring the accuracy of heart rate monitoring and improving the heart rate calculation speed.

The basic concepts have been described. Obviously, for those skilled in the art, the detailed disclosure may be only an example and may not constitute a limitation to the present disclosure. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of the specification are not necessarily all referring to the same embodiment. In addition, some features, structures, or features in the present disclosure of one or more embodiments may be appropriately combined.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, all aspects of the present disclosure may be performed entirely by hardware, may be performed entirely by software (including firmware, resident software, microcode, etc.), or may be performed by a combination of hardware and software. The above hardware or software may be referred to as “data block”, “module”, “engine”, “unit”, “component”. or “system”. In addition, aspects of the present disclosure may appear as a computer product located in one or more computer-readable media, the product including computer-readable program code.

Moreover, unless otherwise specified in the claims, the sequence of the processing elements and sequences of the present application, the use of digital letters, or other names are not used to define the order of the application flow and methods. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure and aiding in the understanding of one or more of the various embodiments. However, this disclosure may not mean that the present disclosure object requires more features than the features mentioned in the claims. In fact, the features of the embodiments are less than all of the features of the individual embodiments disclosed above.

In some embodiments, the numbers expressing quantities, properties, and so forth, used to describe and claim certain embodiments of the application are to be understood as being modified in some instances by the term “about,” “approximate,” or “substantially.” Unless otherwise stated, “about,” “approximate,” or “substantially” may indicate a ±20% variation of the value it describes. Accordingly, in some embodiments, the numerical parameters set forth in the description and attached claims are approximations that may vary depending upon the desired properties sought to be obtained by a particular embodiment. In some embodiments, the numerical parameters should be construed in light of the number of reported significant digits and by applying ordinary rounding techniques. Although the numerical domains and parameters used in the present application are used to confirm the range of ranges, the settings of this type are as accurate in the feasible range in the feasible range in the specific embodiments.

1. A method for monitoring a heart rate, comprising:
obtaining a first signal, the first signal including a target heart rate signal in a motion state;
obtaining a motion signal corresponding to the motion state;
identifying a second signal with a target frequency from the first signal based on a motion frequency corresponding to the motion signal, the target frequency originating from a linear superposition of the motion frequency and a heart rate frequency corresponding to the target heart rate signal; and
determining the target heart rate signal by processing, based on the motion signal and the second signal, the first signal.

2. The method of claim 1, wherein the obtaining a motion signal corresponding to the motion state includes:
obtaining a filtered signal by performing a filtering operation on the first signal; and
determining the motion signal based on the filtered signal.

3. The method of claim 1, wherein the obtaining a motion signal corresponding to the motion state includes obtaining the motion signal through an acceleration sensor.

4. The method of claim 1, wherein the obtaining a motion signal corresponding to the motion state includes:
obtaining two or more first signals using two or more optical paths; and
determining the motion signal based on the two or more first signals.

5. The method of claim 1, wherein the second signal includes a superimposed signal between the motion signal and the target heart rate signal.

6. The method of claim 5, wherein the superimposed signal includes a non-linear superimposed signal.

7. The method of claim 1, wherein the target frequency is equal to a sum of the motion frequency and the heart rate frequency.

8. The method of claim 1, wherein the target frequency is equal to a difference between the motion frequency and the heart rate frequency.

9. The method of claim 1, wherein the determining the target heart rate signal by processing, based on the motion signal and the second signal, the first signal includes:
determining the target heart rate signal by removing the motion signal and the second signal from the first signal.

10. The method of claim 1, wherein the determining the target heart rate signal by processing, based on the motion signal and the second signal, the first signal further includes:
determining a signal amplitude of the motion signal;
determining whether the signal amplitude is greater than an amplitude threshold; and
in response to determining that the signal amplitude is greater than the amplitude threshold, determining the target heart rate signal by processing, based on the motion signal and the second signal, the first signal.

11. The method of claim 1, wherein the determining the target heart rate signal by processing, based on the motion signal and the second signal, the first signal further includes:
determining a signal frequency of the motion signal;
determining whether the signal frequency is greater than a frequency threshold; and
in response to determining that the signal frequency is greater than the frequency threshold, determining the target heart rate signal by processing, based on the motion signal and the second signal, the first signal.

12. The method of claim 1, wherein the target heart rate signal in the motion state included in the first signal is obtained using a photocapacitive pulse wave sensor.

13. A system for monitoring a heart rate, comprising:
at least one storage device storing a set of instructions; and
at least one processor in communication with the storage device, wherein when executing the set of instructions, the at least one processor is directed to cause the system to:
obtain a first signal, the first signal including a target heart rate signal in a motion state;
obtain a motion signal corresponding to the motion state;
identify a second signal with a target frequency from the first signal based on a motion frequency corresponding to the motion signal, the target frequency originating from a linear superposition of the motion frequency and a heart rate frequency corresponding to the target heart rate signal; and
determine the target heart rate signal by processing, based on the motion signal and the second signal, the first signal.

14. The system of claim 13, wherein to obtain a motion signal corresponding to the motion state, the at least one processor is directed to cause the system to:
obtain a filtered signal by performing a filtering operation on the first signal; and
determine the motion signal based on the filtered signal.

15. (canceled)

16. The system of claim 13, wherein to obtain a motion signal corresponding to the motion state, the at least one processor is directed to cause the system to:
obtain two or more first signals using two or more optical paths; and
determine the motion signal based on the two or more first signals.

17. The system of claim 13, wherein the second signal includes a superimposed signal between the motion signal and the target heart rate signal.

18-20. (canceled)

21. The system of claim 13, wherein to determine the target heart rate signal by processing the first signal based on the motion signal and the second signal, the at least one processor is directed to cause the system to:
determine the target heart rate signal by removing the motion signal and the second signal from the first signal.

22. The system of claim 13, wherein to determine the target heart rate signal by processing the first signal based on the motion signal and the second signal, the at least one processor is directed to cause the system to:
determine a signal amplitude of the motion signal;
determine whether the signal amplitude is greater than an amplitude threshold; and
in response to determining that the signal amplitude is greater than the amplitude threshold, determine the target heart rate signal by processing, based on the motion signal and the second signal, the first signal.

23. The system of claim 13, wherein to determine the target heart rate signal by processing the first signal based on the motion signal and the second signal, the at least one processor is directed to cause the system to:
determine a signal frequency of the motion signal;
determine whether the signal frequency is greater than a frequency threshold; and
in response to determining that the signal frequency is greater than the frequency threshold, determine the target heart rate signal by processing, based on the motion signal and the second signal, the first signal.

24. (canceled)

25. (canceled)

26. A non-transitory computer readable medium, including executable instructions that, when executed by at least one processor, cause the at least one processor to perform a method for monitoring a heart rate, the method comprising:
obtaining a first signal, the first signal including a target heart rate signal in a motion state;
obtaining a motion signal corresponding to the motion state;
identifying a second signal with a target frequency from the first signal based on a motion frequency corresponding to the motion signal, the target frequency originating from a linear superposition of the motion frequency and a heart rate frequency corresponding to the target heart rate signal; and
determining the target heart rate signal by processing, based on the motion signal and the second signal, the first signal.
